(19) United States
(12) Patent Application Publication
(10) Pub. No.: US 2024/0207326 A1
(43) Pub. Date: Jun. 27, 2024

(54) COMPOSITION COMPRISING EGG YOLK AND BURNT CERAL POWDER FOR WOUND HEALING

(71) Applicant: Sahebi Aps, Horsens (DK)

(72) Inventor: Sükrüye Sahebi, Horsens (DK)

(21) Appl. No.: 17/913,812

(22) PCT Filed: Feb. 25, 2021
(86) PCT No.: PCT/DK2021/050054

§ 371 Date: Sep. 22, 2022

(30) Foreign Application Priority Data
Feb. 26, 2020 (DK) PA 2020 70122

(51) Int. Cl. A61K 35/57; A61K 9/00; A61K 9/19; A61K 36/899; A61P 17/02

(52) U.S. Cl. CPC A61K 35/57; A61K 9/0014; A61K 9/0017; A61K 9/19; A61K 36/899; A61P 17/02

(57) ABSTRACT

The present invention relates to wound healing compositions, such as topical wound healing compositions and methods of their preparation. In particular, the present invention relates to a composition comprising egg yolk and burned cereal powder. The composition is suitable for wound management in humans or animals.

### FIELD OF THE INVENTION

The present invention relates to wound healing compositions, such as topical wound healing compositions and methods of their preparation. In particular, the present invention relates to a composition comprising egg yolk and burned cereal powder. The composition is suitable for wound management in humans or animals.

### BACKGROUND OF THE INVENTION

Wound healing is a complicated process including inflammation, re-epithelialization, granulation, neovascularization and wound contraction. Several biochemicals are involved in the healing process including antioxidants, cytokines and liver and kidney damage biomarkers. Although several preparations are available for the management of wounds, there is still a necessity for efficacious medicine, preferably low-cost medicine, and/or alternative medicine.

Honey has been used since ancient times to aid wound healing and may be beneficial in first- and second-degree burns, and there may be moderate evidence that honey helps heal partial thickness burns. Traditionally, Aloe vera has also been used, which may also be beneficial in reducing pain. It is believed that in low-income countries, burns are treated up to one-third of the time with traditional medicine.

In Azadi et al. (2015) Jundishapur J Nat Pharm Prod. 2015; 10(1):e20670 “Comparing the Effect of Visceral Fat and Barley Seed Ash (Hordeum Vulgare L) With Silversulfadiazine on Burn Wound Healing in Rats”, a traditional treatment of skin burns is compared with a conventional medicinal treatment.

DE 3933539 and US 219544 concern the use of egg yolk oil in the treatment of burn wounds.

GB 1456276 discloses a process for preparing an ointment comprising admixing olive oil and Achillea millefolium with lime water, separating the resulting admixture into an aqueous phase and a residue, and admixing the residue with egg yolk.

Accordingly, there is a need for alternative compositions and methods for wound treatment, such as treatment of burn wounds or other skin wounds, providing similar or better healing than conventional treatment with e.g. Aloe vera or honey.

### SUMMARY OF THE INVENTION

The present invention was made in view of the prior art described above, and the object of the present invention relates to alternative wound healing compositions, including methods of their manufactures and uses thereof.

Thus, in a first aspect, the present invention provides compositions comprising egg yolk and burned cereal powder, such as a wound healing composition comprising 50-80% per weight egg yolk and 20-50% per weight burned cereal powder.

In a second aspect, the present invention relates to uses of a composition according to the first aspect, such as use as a medicament for wound management, and/or treatment. In some embodiments, the wound composition can be used in post-operative wound treatment and/or management. In some embodiments, said composition can be used as cosmetical and/or skin-care product.

In a third aspect, the present invention pertains to a method for providing a composition according to the first aspect, said method comprising the steps of:
a) burning a cereal under conditions adapted to provide a burned cereal, such as by the use of an open flame;
b) grinding the burned cereal to provide a fine powder, such as powder with a consistency similar to fine ground coffee powder;
c) mixing the fine powder with egg yolk, e.g. by stirring with a spoon, to provide a composition of a consistency suitable for application on a wound, such as a consistency comparable to molten dark chocolate; and optionally
d) storing the composition provided in step (c) under refrigeration or frozen until further use.

In a fourth aspect, the present invention concerns a method for treatment of a wound of a subject, said method comprising the steps of:
a) cleaning the wound (optional); said cleaning may comprise e.g. removal of dead tissue and/or disinfection;
b) applying a layer of around 0.5 to 5 mm of a composition according to the first aspect of the invention or provided according to the third aspect of the invention, on said wound;
c) repeating step (b) if needed;
d) allowing the composition to air-dry, whereupon a protective crust is formed; optionally
e) applying a thin, non-airtight protective layer of a fabric with a loose, open weave such as cotton gauze used for dressing wounds; and optionally
f) applying olive oil or one or more other skin care product(s) around the area to be treated, and/or on the regenerated skin.

In a fifth aspect, the present invention pertains to a composition according to the first or provided according to the third aspect, for use according to the second aspect.

In a sixth aspect, the present invention concerns a receptacle comprising a composition according to the first aspect and/or a composition provided according to the third aspect.

In a seventh aspect, the present invention concerns a kit comprising a receptacle according to the sixth aspect, and a packaging, and optionally, an instruction for use.

In summary, the present invention provides alternative methods and compositions for wound treatment, such as treatment of burn wounds or other skin wounds, providing similar or better healing than conventional treatment with e.g. Aloe vera or honey. Furthermore, in some embodiments, a similar or even better healing may also be provided compared to conventional medical wound treatment.

### BRIEF DESCRIPTION OF THE DRAWING

Embodiments of the invention will be described in the figures:

FIG. 1 shows a bird with an open skin wound on the neck.

FIG. 2 shows wound healing composition applied to an open skin wound.

FIG. 3 shows formation of a crust after several days.

FIG. 4 shows regrowth of features indicating successful wound healing.

FIG. 5 shows a burn wound (female patient, upper thigh).

FIG. 6 shows a female patient after application of the wound healing composition on the thighs.

FIG. 7 shows a female patient after treatment—some discoloration is still visible.

### DETAILED DESCRIPTION OF THE INVENTION

### Definitions

In the context of the present invention, the singular form of a word may include the plural, and vice versa, unless the context clearly dictates otherwise. Thus, the references “a,” “an” and “the” are generally inclusive of the plurals of the respective terms. For example, reference to “an ingredient” or “a method” may include a plurality of such “ingredients” or “methods.”

Similarly, the words “comprise,” “comprises,” and “comprising” are to be interpreted inclusively rather than exclusively. Embodiments provided by the present disclosure may lack any element that is not specifically disclosed herein. Thus, a disclosure of an embodiment defined using the term “comprising” is also a disclosure of embodiments “consisting essentially of” and “consisting of the disclosed components”. Where used herein, terms like “for example”, “e.g.” or “such as”, particularly when followed by a listing of terms, is merely exemplary and illustrative, and should not be deemed to be exclusive or comprehensive. Any embodiment disclosed herein may be combined with any other embodiment disclosed herein.

Unless expressed otherwise, all percentages expressed herein are by weight of the total weight of the composition.

As used herein, “about” is understood to refer to numbers in a range of numerals, for example the range of +/−10, +/−5, +/−2, +/−1, +/−0.5, +/−0.1% of the referenced number. Moreover, all numerical ranges herein should be understood to include all integers, whole or fractions, within the range. “About” may also indicate the variations and/or uncertainties customary in the field.

The phrase “in some embodiments” can be used interchangeably with “in one or more embodiments”.

A “subject” can e.g. be a human or an animal, such as a mammal, bird, reptile, husbandry, or pet. The present disclosure should not be construed as being limited to a specific animal, human or demography.

Examples of animals may comprise mammals, farm animals, husbandry, and/or pets. An animal may also be a bird or a reptile. The term “pet” means an animal kept primarily for company, protection or entertainment of a human. For example, a pet can be an avian, bovine, canine, equine, feline, hircine, lupine, murine, ovine, or porcine animal, but the pet can also be a reptile.

The term “bird” comprises a group of warm-blooded vertebrates constituting the class Aves, commonly characterized by feathers, toothless beaked jaws, the laying of hardshelled eggs, and wings, whose development may vary according to species. Most birds can fly, with the exception of flightless birds, such as penguin, ostrich, kiwi, and emu. A bird can be e.g. be selected from poultry, Galloanserae (fowl), Galliformes, domestic bird, chicken, duck, goose, quail, turkey, or ostrich.

Among egg-laying animals such as birds, the “egg yolk” or “yolk”, also known as “vitellus”, is the nutrient-bearing portion of the egg. Its primary function is believed to be supply nutrients for the development of the embryo.

“Egg yolk oil”, also known as “egg oil” or “ovum oil”, usually derived from the yolk of chicken eggs, consists mainly of triglycerides with traces of lecithin, cholesterol, biotin, xanthophylls lutein & zeaxanthin, and immunoglobulins, depending on its manufacturing process. Modern production methods include liquid-liquid extraction using e.g. hexane, petroleum ether, or chloroform. Unlike traditional egg oil produced by heat, solvent extracted product also contains immunoglobulins which are destroyed at higher temperatures.

It may be important to note that egg yolk comprises many further constituents than egg yolk oil, such as the non-extractable constituents. Without wanting to be bound by any theory, it is further believed that the extraction and/or provision process may denature and/or modify some of the constituents. Thus, there are significant differences between egg yolk, and egg yolk oil. Generally, egg yolk oil is considered not suitable in providing a wound treatment composition according to the present invention.

“Egg white”, also called “albumen” or “glair(e)”, is the clear liquid contained within an egg. It is believed that its primary function is to protect the yolk and provide additional nutrition for the growth of the embryo (when fertilized). Egg white often consists primarily of about 90% water and about 10% proteins (including albumins, mucoproteins, and globulins). Unlike the yolk, which is high in lipids (fats), egg white contains almost no fat, and carbohydrate content is less than 1%. Egg white may comprise about 50% of the protein in the egg.

A “cereal” may refer to any cultivated grass grown for the edible components of its grain (botanically, a type of fruit called a caryopsis). “Cereal grains” are commonly composed of the endosperm, germ, and bran. The terms “cereal” and “cereal grain” may be used interchangeably.

Examples of cereals comprise e.g. rice, maize, wheat, barley, sorghum, millet, oats, rye, triticale, fonio, spelt, einkorn, emmer, durum, kamut, and optionally also pseudocereals, including any combination thereof.

Unless indicated otherwise, “burned cereal” refers to a burned cereal grain, such as a cereal provided according to Example 1. “Burned cereal” may also refer to the remaining ash after burning cereal grains.

Edible grains from other plant families, in particular non-grass plant families are referred to as “pseudocereals”. Examples of pseudocereals comprise e.g. amaranth, breadnut, buckwheat (Polygonaceae), chia (Lamiaceae), cockscomb (also called quail grass or soko), pitseed goosefoot, qañiwa, quinoa (Amaranthaceae) and wattleseed (also called acacia seed).

“Bran”, also known as “miller's bran” is the hard-outer layers of cereal grain. It is believed to comprise the combined aleurone and pericarp. Rice bran is e.g. a by-product from the production of white rice from brown rice.

“Refined grains” or “refined cereals” may refer to a processed cereal, wherein parts of the cereal are removed, such as the bran and/or germ has been removed, e.g. by a milling process. White rice is an example of a refined brown rice. Usually, refined grains comprise a significantly higher carbohydrate content than non-processed cereals.

Commonly, “rice” is the seed of the grass species Oryza sativa (Asian rice) or Oryza glaberrima (African rice). Rice can e.g. be white rice (usually short grain, but can also be long grain, broken rice, basmati, or jasmine rice), brown rice or black rice. The rice can also be suitable for making rice pudding, which usually is a refined, white rice.

In the context of the present invention, a “wound” refers to a type of injury which usually happens relatively quickly in which skin is burned, torn, cut, punctured (an open wound), and/or or where blunt force trauma causes a contusion (a closed wound). A “wound” may also refer to an injury which damages the epidermis of the skin. A “wound” can e.g. be (i) a “clean wound”, such as a wound made under sterile conditions, where there are no or very few organisms present, and the skin is likely to heal without complications; a (ii) “contaminated wound”—usually resulting from accidental injury, and where there are pathogenic organisms and often foreign bodies in the wound; (iii) an “infected wound”—the wound has pathogenic organisms present and multiplying and usually exhibiting clinical signs of infection, such as yellow appearance, soreness, redness, oozing pus and the like; or (iv) a “colonized wound”—such as a chronic situation, containing pathogenic organisms, difficult to heal, e.g. bedsore.

In the context of the present invention, a wound caused by freezing can also be called “frost bite”.

In some embodiments, a “wound” can be the result of a medical treatment and/or intervention, such as surgery.

Furthermore, a wound can be caused intentionally (e.g. surgery), or accidentally.

The term “wound” may also comprise:
Incisions or incised wounds—often caused by a clean, sharp-edged object such as a knife, razor, or glass splinter;
Lacerations—irregular tear-like wounds caused by some blunt trauma. Lacerations and incisions may appear linear (regular) or stellate (irregular);
Abrasions or grazes—superficial wounds in which the topmost layer of the skin (the epidermis) is scraped off. Abrasions are often caused by a sliding fall onto a rough surface such as asphalt, tree bark or concrete;
Avulsions—injuries in which a body structure is forcibly detached from its normal point of insertion. A type of amputation where the extremity is pulled off rather than cut off.

When used in reference to skin avulsions, the term ‘degloving’ is also sometimes used as a synonym;
Puncture wounds—caused by an object puncturing the skin, such as a splinter, nail or needle;
Penetration wounds—caused by an object such as a knife entering and coming out from the skin; and
Gunshot wounds—caused by a bullet or similar projectile driving into or through the body. There may be two wounds, one at the site of entry and one at the site of exit, generally referred to as a “through-and-through.”
Burn wounds—A “burn wound” or “burn” is a type of injury to skin, or other tissues, caused by heat, cold, electricity, chemicals (e.g. corrosive chemicals), friction, or radiation (e.g. sunlight and/or UV light). Most burns are due to heat from hot liquids, solids, fire or chemicals. Burns that affect only the superficial skin layers are known as superficial or first degree burns. They appear red without blisters and pain typically lasts around three days. When the injury extends into some of the underlying skin layer, it is a partial-thickness or second degree burn. Blisters are frequently present, and they are often very painful. Healing can require up to eight weeks and scarring may occur. In a full-thickness or third degree burn, the injury extends to all layers of the skin. Often there is no pain and the burnt area is stiff. Healing typically does not occur on its own. A fourth degree burn additionally involves injury to deeper tissues, such as muscle, tendons, or bone. The burn is often black and frequently leads to loss of the burned part.
Frost bite(s)—Such wound(s) can be caused by freezing, such as wounds caused by exposure of skin/body parts to low temperatures, causing freezing of skin and/or other tissues. Usually, such wounds can e.g. be caused by cold air, usually in combination with inadequate protective clothing, contact with liquid gas (e.g. CO₂, N₂, or the like), or contact with frozen solid matter, such as metal surfaces or the like. These can be classified and/or described as follows: First degree frostbite is superficial, surface skin damage that is usually not permanent. Early on, the primary symptom is loss of feeling in the skin. In the affected areas, the skin is numb, and possibly swollen, with a reddened border. In the weeks after injury, the skin's surface may slough off. In second degree frostbite, the skin develops clear blisters early on, and the skin's surface hardens. In the weeks after injury, this hardened, blistered skin dries, blackens, and peels. At this stage, lasting cold sensitivity and numbness can develop. In third degree frostbite, the layers of tissue below the skin freeze. Symptoms include blood blisters and “blue-grey discoloration of the skin”. In the weeks after injury, pain persists, and a blackened crust (eschar) develops. There can be long-term ulceration and damage to growth plates. In fourth degree frostbite, structures below the skin are involved like muscles, tendon, and bone. Early symptoms include a colourless appearance of the skin, a hard texture, and painless rewarming. Later, the skin becomes black and mummified. The amount of permanent damage can take one month or more to determine.

In a first aspect, the present invention concerns a composition comprising egg yolk and burned cereal powder. Such a composition can e.g. be used for wound treatment as disclosed elsewhere herein. It may thus also be referred to “composition for wound treatment” or “wound healing composition”. Details and/or embodiments concerning the provision of the wound healing composition can e.g. be found in the Examples, such as Examples 1-3, 9 and 10, as well as in the section of the description concerning e.g. the third aspect of the invention.

In one or more embodiments, a composition is provided comprising at least 80, 85, 90, 95, 98, or 99% per weight egg yolk and burned cereal powder. Thus in some embodiments, one or more further components may be present, such as up to 20, 15, 10, 5, 2, 1% or less per weight of the combined composition.

Usually, the composition comprises around 50-80% per weight egg yolk and 20-50% per weight burned cereal powder. In some embodiments, the composition comprises 60-70% per weight egg yolk and 30-40% per weight burned cereal powder. In some embodiments, the composition comprises around 65% per weight egg yolk and 35% per weight burned cereal powder. Such ratios are believed to provide a composition with a texture suitable for application to a wound as described herein.

In some embodiments, when the composition comprises one or more further components, said ranges (i.e. 50-80% per weight egg yolk and 20-50% per weight burned cereal powder, 60-70% per weight egg yolk and 30-40% per weight burned cereal powder, or around 65% per weight egg yolk and 35% egg yolk) can be pr. total weight of the composition. In some embodiments, said ranges can be based on the combined weight of egg yolk and burned cereal, thus excluding the one or more further component.

Egg yolk can be separated from egg white using common techniques known in the art. Egg yolks can also be purchased e.g. in a supermarket, often also as “salmonella-free” egg yolks. Likewise, salmonella-free eggs can be used. Thus in some embodiments, the composition comprises salmonella-free egg yolk.

A common source for eggs and thus also egg yolk is chicken eggs. However, it is believed that eggs from other birds can be used as well. In some embodiments, the egg yolk is from a bird selected from poultry, Galloanserae (fowl), Galliformes, domestic bird, chicken, duck, goose, quail, turkey, or ostrich. In a preferred embodiment, the egg yolk is from a chicken. Generally, it is believed that egg yolk cannot be substituted by—or replaced in full with—egg yolk oil without negative impact on the effect and/or on the composition's effect on wound healing.

Often, a separation of egg yolk and egg white may not be 100%, thus a minor amount of egg white can still be present, such as around the yolk. Thus, in some embodiments, the yolk comprises around 1, 2, 3, 4, 5, 7 or 10% egg white. In some embodiments, the yolk comprises around less than 1, 2, 3, 4, 5, 6, 7, 8, 9, 10, 11, 12, 13, 14, 15, 16, 17, 18, 19 or 20% egg white. In some embodiments, the yolk comprises around 0.1-2, 2-4, 4-6, 6-8, 8-10, 10-15, or 15-20% egg white.

In some embodiments, the yolk comprises 0.1-10, or 0.1-20% egg white, such as around 0-5, 5-10, or 10-20% egg white.

Generally, a higher content of egg white may not be desirable, as it may lead to one or more undesired features, such as texture, consistency during or upon manufacture. Furthermore, elevated egg white concentrations may also lead one or more undesired effects upon application, such as smell, microbial growth, slower healing, and increased risk of infections, reduced storability or the like.

In some embodiments, the cereal comprises one or more pseudocereals. In some embodiments, the cereal does not comprise one or more pseudocereals—this may also be referred to as “non-pseudocereal”.

In some embodiments the composition comprises burned cereal powder, wherein the cereal is selected from one or more of rice, maize, wheat, barley, sorghum, millet, oats, rye, triticale, fonio, spelt, einkorn, emmer, durum, kamut, and optionally also pseudocereal(s), including any combination thereof. Thus the cereal may comprise a mixture of different cereals, different pseudocereals, or a mixture of one or more cereal with one or more pseudocereal.

In some embodiments, the cereal is a pseudocereal, such as one or more of amaranth, breadnut, buckwheat (Polygonaceae), chia (Lamiaceae), cockscomb (also called quail grass or soko), pitseed goosefoot, qaniwa, quinoa (Amaranthaceae) and wattleseed (also called acacia seed), including any combination thereof.

In some embodiments, the burned cereal is provided from one species or type of cereal. In some embodiments, the burned cereal is provided from two or more species or types of cereals. This may also include a mixture of one or more non-pseudocereals with one or more pseudocereal.

In some embodiments, the cereal is or comprises a hull-less cereal, a de-hulled cereal or a refined cereal. In some embodiments, the cereal comprises bran. In some embodiments, the cereal does not comprise bran.

In some embodiments, the cereal is or comprises rice, such as de-hulled or refined rice. In some embodiments, the rice is the seed of the grass species Oryza sativa (Asian rice) or Oryza glaberrima (African rice). In some embodiments, the rice is e.g. white rice (usually short grain, but can also be long grain, broken rice, basmati, or jasmine rice), brown rice or black rice. In some embodiments, the rice is suitable for making rice pudding.

In some embodiments, the wound healing composition comprises egg yolk from chicken, and/or burned rice. In some embodiments, the composition consists essentially of egg yolk, such as egg yolk from chicken, and burned rice.

As disclosed herein, a composition according to the present invention can be used for skin treatment. In some embodiments, the composition is for topical use, e.g. for skin treatment, such as treatment of a wound of a subject.

In some embodiments, the composition is suitable for treatment of skin of a subject, such as a wound related to burn, frost bite, bite, abrasion, avulsion, incision, laceration, puncture, penetration, radiation (e.g. UV radiation and/or sun light) and/or gunshot. Furthermore, in some embodiments, the wound can also be a clean wound, contaminated wound, infected wound or colonized wound.

In some embodiments, the subject is a human and/or an animal, such as a mammal, bird, reptile, husbandry, and/or pet.

In some embodiments, and in contrast to prior art compositions, a composition according to the first aspect does not comprise one or more of: visceral fat, plant oil, and/or egg yolk oil, including any combination thereof.

In a second aspect, the present invention relates to the use of a composition according to the first aspect, such as any one of the embodiments according to the first aspect as a medicament, cosmetics and/or skin-care product.

In some embodiments, said uses can be a use as a medicament, such as a topical composition. In some embodiments, said composition can e.g. be used as a facilitator for wound treatment or wound healing, and/or as a medicament for wound treatment or wound healing. In some embodiments, said composition can be used as a medicament, such as a medicament for wound treatment. In some embodiments, said composition is suitable and/or used for wound treatment.

A wound can be any wound as disclosed herein. In some embodiments, the wound is related to one or more of: burn, frost bite, bite, abrasion, avulsion, incision, laceration, puncture, penetration, gunshot, exposure to chemicals, and/or surgery. Exposure to chemicals may comprise acids, basis, and/or other chemicals, harmful for to the skin of a user, such as corrosive chemicals.

In some embodiments, wound is a burn wound. In some further embodiments, the burn or burn wound is a 1^{st}, 2^{nd}, 3^{rd}, or 4th degree burn or burn wound. In some embodiments, the burn or burn wound is a 1^{st}, 2^{nd} or 3^{rd} degree burn or burn wound. In some embodiments, the wound is related to sunburn and/or exposure to UV light.

In some embodiments, the wound is a frost bite. In some embodiments, the wound is a 1^{st}, 2^{nd}, 3^{rd}, or 4th degree frost bite. In some embodiments, the wound is a 1^{st}, 2^{nd} or 3^{rd} degree frost bite.

In some embodiments, the wound is a result of a medical and/or cosmetical intervention, such as surgery. In some embodiments, the composition can be used for post-operative wound treatment.

In some embodiments, the use or treatment of a wound may comprise applying a layer of around 0.5 to 5 mm of said composition on said wound. Thereto, it can be advisable to use a wound healing composition with a suitable consistency or texture, such that the composition can be applied with minimal discomfort for the subject, and/or that e.g. dripping is avoided.

A suitable consistency/texture of said composition can e.g. be comparable to molten dark chocolate, toothpaste, mayonnaise, Nivea cream or the like. A suitable consistency can also be described as suitable to be applied on skin wound with e.g. a spatula. This may imply that the composition is not too thin or liquid. This may also imply that the composition is not too solid, thus not allowing for applying a uniform layer, preferably with minimal discomfort for the patient to be treated. A suitable consistence can also refer to a consistency or texture that allows for formation of a crust upon application and air drying, such as after 5 or 10 min.

It is believed that it is a further advantage of the wound treatment composition that a crust is provided upon application and air drying. Without wanting to be bound by any theory, it is believed that said crust improves and/or contributes to the healing process, and/or provides a further protection of the wound against e.g. contamination, disturbance of the wound healing process and/or other risks. In some embodiment, said use comprises the provision of a protective crust. In some embodiments, said crust is provided upon air-drying of wound healing composition. In this context, in some embodiments, the use of egg yolk instead of egg yolk oil is considered important in providing the appropriate texture and effect of the composition, comprising e.g. the formation of the protective crust.

In a third aspect, the present invention pertains to a method for providing a composition according to the first aspect. In some embodiments, such a method comprises may comprise the acts (or steps) of:
i. burning a cereal under conditions adapted to provide a burned cereal, such as by the use of an open flame;
ii. grinding the burned cereal to provide a powder, such as powder with a consistency similar to fine ground coffee;
iii. mixing the fine powder with egg yolk, e.g. by stirring with a spoon, to provide a composition of a consistency suitable for application on a wound, such as a consistency comparable to molten dark chocolate; and optionally
iv. storing the composition provided in step (iii) under refrigeration or frozen until further use.

In some embodiments, said method may further comprising a conservation step (v). Said conservation step may comprise refrigeration, freezing, freeze drying and/or provision of a protective atmosphere. In some embodiments, a freeze-dried composition is resuscitated with water, such as distilled water, before use.

In some embodiments, said method may comprise, or may consist essentially of the steps (or acts) of:
a) providing a cereal, e.g. rice;
b) providing egg yolk; e.g. egg yolk form chicken;
c) burning said cereal under conditions adapted to provide a burned cereal, such as by the use of an open flame to ignite the cereal;
d) grinding the burned cereal to provide a fine powder, such as powder with a consistency similar to fine ground coffee; optionally
e) sieving the burned cereal powder to provide a uniform powder; and
f) mixing the fine powder with egg yolk, e.g. by stirring with a spoon, to provide a composition with a consistency suitable for application onto a wound, such as a consistency comparable to molten dark chocolate, toothpaste, mayonnaise, or Nivea cream; and optionally
g) storing the composition provided in step (f), such as under refrigeration until further use.

In some embodiments, step (d) comprises the use of a mortar and pestle. In some embodiments, step (d) comprises the use of a grinder or mill, such as a household coffee grinder.

In some embodiments, the sieving in step (e) is conducted using a fine household sift.

In some embodiments, storage is performed by refrigeration at around 2-8° C., or around 4-6° C. In some embodiments, refrigeration is performed in a household refrigerator. In some embodiments, storage is performed around 0° C., such as around −4 to +4° C., or around −2 to +2° C. In some embodiments, the wound healing composition is placed in an appropriate receptacle, such as a container, e.g. a container with a lid, such as a reclosable and/or resealable container for storage. In some embodiments, the composition can also be frozen, such as at temperatures below −16, −18, −20° C. or below, such as in a conventional household freezer. When stored at around −18° C., the composition may not be frozen completely, and may maintain a consistency comparable to e.g. ice cream stored at these temperatures. In some embodiments, the composition can be stored frozen for one or more weeks, one or more month, or even half a year or more. It is believed that thawing can be performed using common techniques known in the art, such as placing the frozen composition in a refrigerator, thawing at room temperature, or in a water bath. In some embodiments, thawing is performed at a temperature below room temperature, such as at or below around 12, 10, 8, 6, 4, or 2° C. It may be advisable to stir the composition with e.g. a spoon or spatula before use after a prolonged storage period.

In some embodiments, said method may further comprise a conservation step. Said conservation step may comprise refrigeration and/or freezing. In some embodiments, said conservation step may comprise cold storage (refrigerated or frozen) and/or freeze-drying. In some embodiments, the wound treatment composition can be freeze-dried for prolonged storage. In some embodiments, the freeze-dried composition is stored under vacuum or reduced pressure. In some embodiments, the freeze-dried composition is stored under a protective atmosphere, such as a gas without oxygen, such as N₂ or CO₂. Storage under vacuum, reduced pressure and/or protective atmosphere is believed to improve storage of the freeze-dried composition, such as avoiding oxidation, rancidification, or the like; and/or growth of undesired microorganisms.

The freeze-dried composition can be resuscitated upon addition of a resuscitation medium, comprising essentially water. In some embodiments, the freeze-dried composition can be stored for prolonged periods, such as for at least half a year, one year, or more than 1 or 2 years without refrigeration, such as at room temperature, e.g. at 18-30° C., or 20-25° C. In some embodiments, the resuscitation medium is distilled water. In some embodiments, the resuscitation medium may comprise a further agent. Generally, freeze-drying and/or resuscitation of freeze-dried composition according to the invention can be performed with general methods known in the art (see also e.g. Examples 9 and 10).

Further details and/or embodiments concerning the provision of the wound healing composition can e.g. be found in the Examples, such as Examples 1-3.

Depending on the kind of wound and/or the subject to be treated, it may be advisable to work under as hygienic conditions as possible, e.g. using sterile or disinfected equipment and/or compositions.

In a fourth aspect, the present invention concerns a method for treatment of a wound of a subject, said method comprising the steps of:
a) cleaning the wound (optional);
b) applying a layer with a thickness of around 0.5 to 5 mm of a wound healing composition on a wound, such as a composition according to the first aspect, and/or provided according to the third aspect of the invention;
c) repeating step (b) if needed;
d) allowing the composition to air-dry, whereupon a protective crust is formed; optionally
e) applying a thin, non-airtight protective layer of a fabric with a loose, open weave such as cotton gauze used for dressing wounds, and/or optionally
f) applying olive oil and/or one or more other skin care product(s) around the area to be treated, and/or on the regenerating and/or regenerated skin.

Cleaning of the wound may comprise removal of foreign objects, and/or removal of dead skin or tissue. Said cleaning may also comprise disinfection. It is believed to be advisable to remove dead skin/tissue in order to increase adhesion of the wound healing composition to the wound, such as the ‘crust’.

In some embodiments, the wound is the result of a medical intervention, such as a surgical procedure. This may comprise deeper cuts, e.g. cuts through skin and muscle tissue, or less deeper cuts, such as predominantly through skin tissue. In some embodiments, such a wound may further comprise e.g. staples and/or sutures or the like. In some embodiments, the medical intervention may comprise freezing.

In some embodiments, the wound treatment is or comprises post-operative wound management.

Application of olive oil and/or other skin care products in combination with the wound healing composition as disclosed herein is believed to contribute to the healing process, and/or to reduce discomfort for the subject to be treated. This may comprise reduction of “itchiness” around the wound, and/or the regenerated or regenerating tissue, such as where the crust as broken loose, and said regenerated tissue is exposed.

In some embodiments, without wanting to be bound by any theory, it is believed that application of the wound composition improves relaxation of the subject, such as enabling or improving sleeping. Generally, alleviation of symptoms may comprise one or more of: reduced pain, reduced sensitivity to touch, reduced discomfort, such as reduced itching, burning, and/or stinging sensation(s).

In some embodiments, the subject to be treated is recommended to rest for at least 6, 8, 10, 12 or more hours after treatment with the wound healing composition.

Sometimes, a fluid may appear upon application of the wound healing composition. Suitably, said fluid is carefully removed, e.g. by a clean cloth or tissue. It may be advisably to apply more wound composition afterwards.

Suitably, any cracks or openings in the “crust” can be filled and/or covered with more of the wound healing composition. It may be necessary to repeat this process several times, until the wound is healed.

Generally, use of a wound healing composition as disclosed herein, is believed to provide one or more of the following effects: improved wound healing, less scar tissue, less complications, and/or less discomfort during healing process, compared to conventional treatment, which may comprise alternative or conventional medical treatment. This can be especially important with respect to wound treatment concerning parts of the skin that are usually not protected by clothing, such as face, neck, arms, legs and the like. In some embodiments, said effects are related to post-operative treatment, wherein the use of the wound healing composition provides less scaring. In some embodiments, the wound healing composition may also be used for reducing post-operative scars.

In a fifth aspect, the present invention pertains to a composition according to the first aspect and/or a composition provided according to the third aspect, for use according to the second aspect. Such uses may comprise one or more use(s) as: medicament, topical composition, composition for wound treatment, post-operative wound management, cosmetical, and/or skin-care product. Further details are given herein, e.g. in the second aspect of the invention.

In a sixth aspect, the present invention concerns a receptacle comprising a composition according to the first aspect and/or a composition provided according to the third aspect.

In a seventh aspect, the present invention concerns a kit comprising a receptacle according to the sixth aspect, and a packaging, and optionally, an instruction for use. In some embodiments, said kit may comprise a receptacle with a freeze-dried composition and a further receptacle with a resuscitation liquid, such as distilled water.

Further details and/or embodiments concerning the wound healing composition including e.g. methods of manufacture as well as their application in treatment of animals and/or humans can e.g. be found in the following Examples.

### EXAMPLES

### Example 1

### Provision of Burned Cereal Powder

100 g cereal, e.g. rice, such as rice for rice pudding, is burned in an open fireproof container, such as a metal pot filled with a couple of cm of rice, using an open flame, such as a flame from a weed burner to ignite the rice. The burning cereal is stirred occasionally, and the weed burner is used repeatedly to ensure re-igniting and burning of the cereal. This process is terminated once no more unburned cereal is visible to the naked eye, and when the cereal fails to burn any further. This may be after around 5-10 min.

The burned cereal ash is ground to a fine and uniform powder by the use of grinding means, such as an electric household coffee grinder, or a mortar and pestle. A suitable consistency of the burned cereal powder can e.g. be fine-ground coffee, such as for espresso or filter coffee. If needed, a mesh strainer or sift can be used to provide a more uniform burned cereal powder by sieving. It is advisable to disinfect grinding or sieving means prior use, such as with 70% ethanol or the like.

### Example 2

### Provision of Egg Yolk

Salmonella-free yolk can be obtained in a supermarket. Alternatively, fresh eggs can be used, where the egg yolk is separated from the egg white. This can e.g. be done as follows: Cracking an egg, e.g. by tapping the egg on a flat surface, such as the countertop, to crack open its shell, preferably obtaining two shell halves. Let the yolk settle in one of the open halves. Transfer the yolk back and forth between the two halves, by carefully pouring the yolk into the other half of the shell, letting the egg white drip into a bowl or the like. Repeat this step, shifting the yolk from one shell to the other, no more, or only very little egg white can be removed. Then carefully pour the yolk into suitable container, if not used shortly thereafter. After separation, the egg yolk can be stored in a refrigerator at around 4-6° C. for a couple of days before use.

### Example 3

### Provision of a Wound Healing Composition

Around 35 g of burned cereal powder (Example 1) is combined with around 65 g egg yolk (Example 2) and carefully mixed to a fine paste/cream by stirring with a spoon, until a suitable consistency is provided. If needed, more burned cereal powder can be added, if the consistency is too thin/liquid or vice versa, more egg yolk. A suitable consistency can e.g. be comparable to molten dark chocolate, toothpaste, mustard and/or Nivea cream.

The wound healing composition can be stored refrigerated (e.g. 4-6° C.) for some days before use. It is advisable to store the composition in a container that allows some stirring in case that some precipitation has occurred.

### Example 4

### Application of the Wound Healing Composition

The wound healing composition according to Example 3 is applied to the wound and surrounding area e.g. with a clean spatula in a sufficient thickness, such as e.g. around 0.5-5 mm, and allowed to air-dry, whereupon a solid crust-like surface is formed. If any fluid appears, said fluid is carefully removed, e.g. by a clean cloth or tissue. Any cracks or openings are filled and covered with more of the wound healing composition. It may be necessary to repeat this process several times, until the wound is healed. It can furthermore be advisable to clean the wound before application of the wound healing composition, such as by the use of methods and compositions customary in the field. Said cleaning may comprise removal of dead tissue in order to improve adhesion of the wound healing composition to the wound.

Sometimes, it can be advantageous to further protect the wound and/or protective crust with a thin, non-airtight protective layer of a fabric with a loose, open weave such as cotton gauze used for dressing wounds.

### Example 5

### Comparison of Treated and Untreated Burn Wounds

In a control experiment, similar burn wounds, such as 1^{st}, 2^{nd} or 3^{rd} degree burn wounds are treated with a wound healing composition according to Example 3+4, and one or more controls. Said control can be “no treatment” or treatment with an alternative remedy, such as application of honey or Aloe vera.

Treatment with the wound healing composition provides better results than the “no treatment” control, and similar or better results than the “alternative remedy” control. Similar or better results can be scored with respect on pain relief, healing time, and/or complications (e.g. infections), including any combination thereof.

### Example 6

### Comparison of Treated and Untreated Wounds

In a control experiment, similar wounds, such as abrasion, puncture or incision wounds are treated with a wound healing composition according to Example 3+4, and one or more controls. Said control can be “no treatment” or treatment with an alternative remedy, such as application of honey or Aloe vera.

Treatment with the wound healing composition provides better results than the “no treatment” control, and similar or better results than the “alternative remedy” control. Similar or better results can be scored with respect to pain relief, healing time, and/or complications (e.g. infections), including any combination thereof.

### Example 7

Treatment of Bird with an Open Skin Wound

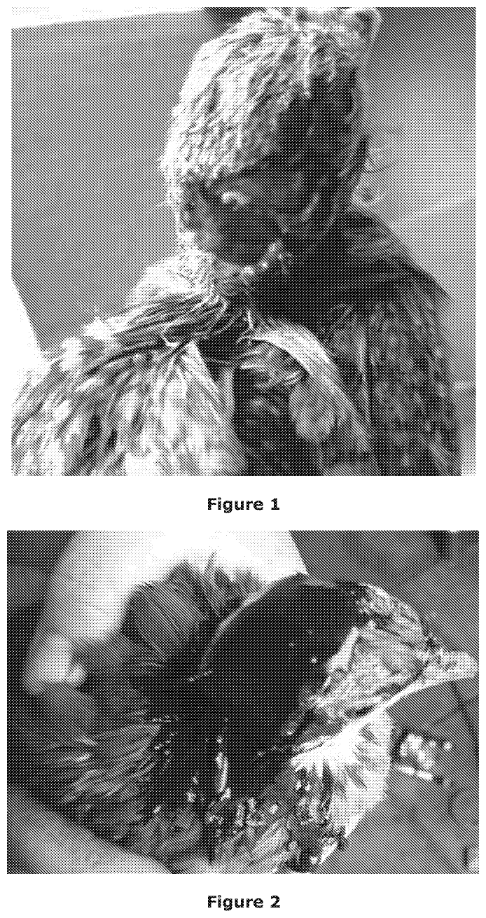

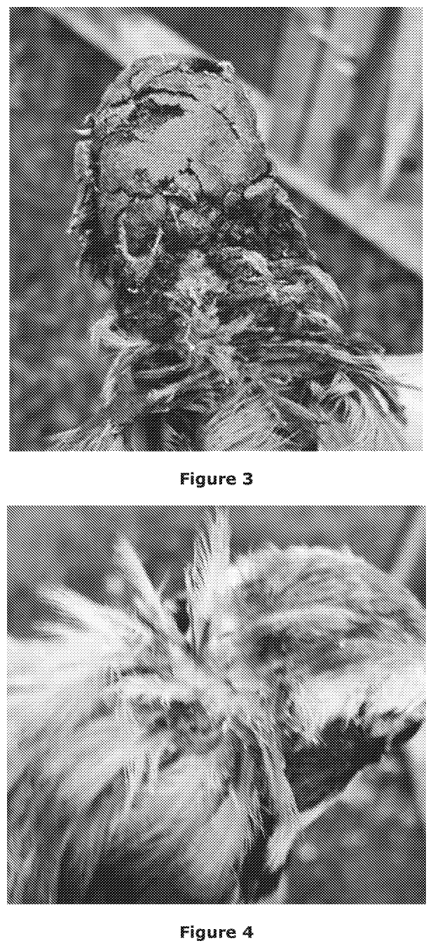

A bird with a nasty open skin wound on the neck (FIG. 1) is treated with a wound healing composition provided according to Example 3. The wound healing composition consists essentially of burned rice powder and chicken egg yolk, and approximately 35 g burned rice powder and approximately 65 g chicken egg yolk. FIG. 2 shows the bird during application of the wound healing composition, before a crust has formed. FIG. 3 shows the same bird after several days—a crust has formed, which has started to fall off during wound healing. FIG. 4 illustrates the same bird, where features started to reappear, indicating that the wound has healed even further.

### Example 8

A Treatment of Female Patient with Burn Wound on Upper Thighs

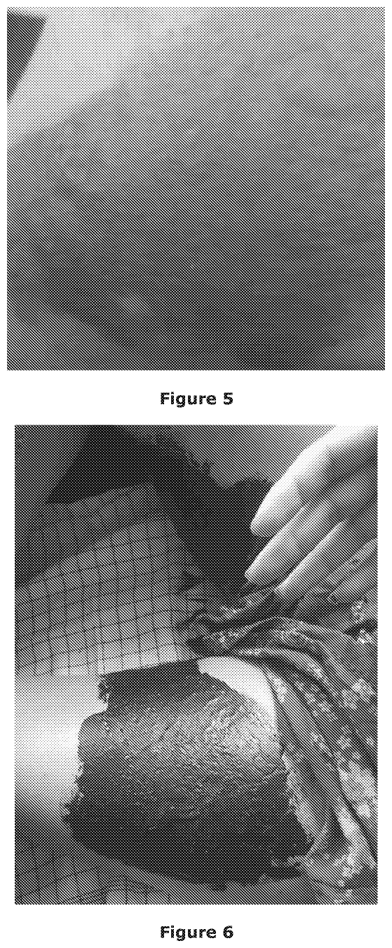

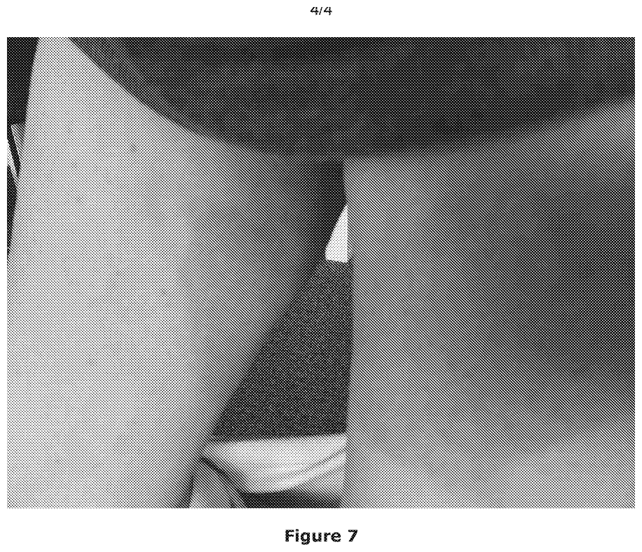

A female patient with burn wounds on both upper thighs caused by boiling water was treated with a wound healing composition provided according to Example 3 and applied according to Example 4. FIG. 5 shows a major part of the burn wound on one of the thighs. Blisters and tissue damage are visible. FIG. 6 shows the female patient after application of the wound healing composition on the thighs. FIG. 7 shows a female patient around 10 days after treatment—some discoloration is still visible. The regenerated skin was treated repeatedly with olive oil.

B Treatment of Patient with Sunburn

A patient with sunburn (red skin, no blisters) was treated with a wound healing composition according to Example 3, and applied according to Example 4. After two days, the skin was completely regenerated, and the patient was free of symptoms.

### Example 9

### Long Term Storage

A wound healing composition provided according to Example 3 was kept frozen for several months. Upon careful thawing in a water bath with cold tap water and after some stirring, the composition could be used in a similar fashion compared to a freshly provided composition.

### Example 10

### Freeze Drying

A wound healing composition provided according to Example 3 is freeze dried using conventional freeze-drying equipment. The freeze-dried composition is stored in a closed and airtight container at room temperature (around 20-25° C.) for 6 months. Upon careful resuscitation with sterile water until the desired consistency is achieved, the composition can be used in a similar fashion compared to a freshly provided composition.

1. A wound healing composition comprising egg yolk and burned cereal powder.

2. A composition according to claim 1, comprising at least 80% per weight egg yolk and burned cereal powder.

3. A composition according to claim 1, comprising 50-80% per weight egg yolk and 20-50% per weight burned cereal powder; 60-70% per weight egg yolk and 30-40% per weight burned cereal powder; or around 65% per weight egg yolk and 35% per weight burned cereal powder.

4. A composition according to claim 1, wherein the egg yolk is salmonella-free.

5. A composition according to claim 1, wherein the egg yolk is from a bird's egg, said bird being selected from poultry, Galloanserae (fowl), Galliformes, domestic bird, chicken, duck, goose, quail, turkey, or ostrich.

6. A composition according to claim 1, said composition comprising less than 20, 10, 5, or 2% per weight egg white.

7. A composition according to claim 1, wherein the cereal is selected from one or more of: rice, maize, wheat, barley, sorghum, millet, oats, rye, triticale, fonio, spelt, einkorn, emmer, durum, kamut, and pseudocereal, including any combination thereof.

8. A composition according to claim 1, wherein the cereal is or may comprise a pseudocereal, such as amaranth, breadnut, buckwheat, chia, cockscomb, pitseed goosefoot, qaniwa, quinoa and wattleseed, including any combination thereof.

9. A composition according to claim 1, wherein the cereal is a hull-less cereal, a de-hulled cereal or a refined cereal.

10. A composition according to claim 1, wherein the cereal is rice, such as de-hulled or refined rice.

11. A composition according to claim 1, wherein the cereal is rice suitable for making rice pudding.

12. A composition according to claim 1, wherein the egg yolk is from chicken, and/or the burned cereal is rice.

13. A composition according to claim 1, for topical use, e.g. for skin treatment, such as treatment of a wound of a subject.

14. A composition according to claim 1 for treatment of skin of a subject, such as a wound related to burn, frost bite, bite, abrasion, avulsion, incision, laceration, puncture, penetration, gunshot, exposure to chemicals, radiation (e.g. sunlight and/or UV light) and/or surgery.

15. A composition according to claim 13, wherein the subject is a human and/or an animal, such as a mammal, bird, reptile, husbandry, or pet

16. Use of a composition according to claim 1 as a medicament, such as a medicament for wound treatment.

17. Use of a composition according to claim 1 for wound treatment, such as post-operative wound management.

18. Use according to claim 16, wherein the wound is related to one or more of: burn, frost bite, bite, abrasion, avulsion, incision, laceration, puncture, penetration, gunshot, exposure to chemicals, radiation and/or surgery.

19. Use according to claim 18, wherein the wound is a burn wound, a frost bite, and/or a wound related to a surgical procedure.

20. Use according to claim 19, wherein the burn is a 1st, 2nd, 3rd or 4th degree burn, or a 1st, 2nd, 3rd or 4th degree frost bite.

21. Use of a composition according to claim 16, comprising applying a layer of around 0.5 to 5 mm of said composition on said wound.

22. Use according to claim 16, comprising the provision of a protective crust.

23. Use according to claim 22, wherein said crust is provided upon air-drying of the composition.

24. A method for providing a composition according to claim 1, comprising the steps of: a. burning a cereal under conditions adapted to provide a burned cereal, such as by the use of an open flame; b. grinding the burned cereal to provide a powder, such as powder with a consistency similar to fine ground coffee; c. mixing the fine powder with egg yolk, e.g. by stirring with a spoon, to provide a composition of a consistency suitable for application on a wound, such as a consistency comparable to molten dark chocolate; and optionally d. storing the composition provided in step (c) under refrigeration or frozen until further use.

25. A method according to claim 24, further comprising a conservation step (e).

26. A method according to claim 25, wherein said conservation step (e) comprises cold storage (refrigerated or frozen) and/or freeze-drying, and optionally storage under vacuum, reduced pressure and/or protective atmosphere.

27. A method for treatment of a wound of a subject, comprising the steps of: a. cleaning the wound (optional); wherein said cleaning may comprise e.g. removal of dead tissue and/or disinfection; b. applying a layer of around 0.5 to 5 mm in thickness of a composition according to claim 1, on said wound; c. repeating step (b) if needed; d. allowing the composition to air-dry, whereupon a protective crust is formed; optionally e. applying a preferably thin, non-airtight protective layer of a fabric with a loose, open weave such as cotton gauze used for dressing wounds on top of the protective crust; and/or optionally f. applying olive oil and/or one or more other skin care product(s) around the area to be treated, and/or on the regenerating or regenerated skin.

28. A composition according to claim 1 or provided according to a method according to claim 24.

29. Composition according to claim 28 for use according to claim 16, such as use as a medical, cosmetical and/or skin-care product.

30. A receptacle comprising a composition according to claim 28.

31. A kit comprising a receptacle according to claim 30 and a packaging and/or an instruction for use.

32. A kit according to claim 31, wherein the composition is freeze-dried, further comprising a receptacle with a resuscitation liquid, such as distilled water.
